(19) United States
(12) Patent Application Publication
(10) Pub. No.: US 2020/0264088 A1
(43) Pub. Date: Aug. 20, 2020

(54) AIR QUALITY POLE MODULE AND LAMP POST COMPRISING SUCH A MODULE

(71) Applicant: Schreder S.A., Bruxelles (BE)

(72) Inventor: Richard John David Bentley, Eastleigh-Hampshire (GB)

(21) Appl. No.: 16/648,205

(22) PCT Filed: Sep. 17, 2018
(86) PCT No.: PCT/EP2018/075070

§ 371 Date: Mar. 17, 2020

(30) Foreign Application Priority Data
Sep. 18, 2017 (BE) 20175657

(51) Int. Cl. G01N 15/06; F21S 8/08; G01N 33/00

(52) U.S. Cl. CPC G01N 15/0656; G01N 2015/0046; G01N 33/0073; F21S 8/088

(57) ABSTRACT

Air quality pole module for a lamp post comprising pole modules arranged one above the other in an axial direction, said air quality pole module comprising: a core support; a cover assembly attached to the core support such that they form a housing; an air quality sensing assembly arranged in said housing and comprising a gas sensor, a particle counter and a control unit for obtaining air quality data based on measurements by said gas sensor and the particle counter; wherein the air quality sensing assembly is provided with an EMI shield with at least one aperture for allowing the passage of air to the gas sensor; wherein the housing is provided with a first opening for allowing an air flow towards the at least one gas sensor and the particle counter, and a second opening connected to the particle counter at a distance of said first opening.

### FIELD OF INVENTION

The field of the invention relates to modular lamp posts, in particular lamp posts in the form of outdoor luminaires. Particular embodiments relate to an air quality pole module for use in such lamp posts.

### BACKGROUND

EP 3 076 073 B1 in the name of the applicant discloses a modular lamp post which is readily assembled and installed in the field whilst providing rigidity, structural integrity and sealing. The lamp post comprises a plurality of modules mounted on a support pole. The modules are connected to one another by respective module connectors and one module thereof is connected to the support pole by a module connector. EP 3 076 073 B1 is included herein by reference.

Further it is known to include air quality monitoring functionalities in separate units attached to a lamp post.

### SUMMARY

The object of embodiments of the invention is to provide an air quality pole module that can be easily integrated in the lamp post, whilst at the same time being capable of performing accurate measurements of the air quality. The object of particular embodiments is to allow integrating air quality monitoring functionalities in lamp posts in an improved manner compared to prior art solutions.

According to a first aspect of the invention there is provided an air quality pole module for a lamp post. The air quality pole module comprises an air quality sensing assembly, and a core support and a cover assembly, which form a housing for the air quality sensing assembly. The core support extends in the axial direction of the pole module between a top end and a bottom end. The cover assembly is coupled to the core support such that the core support and the cover assembly together form the housing for the air quality sensing assembly. The air quality sensing assembly comprises at least one gas sensor, a particle counter and a control unit configured for obtaining and outputting air quality data based on measurements by said at least one gas sensor and the particle counter. The air quality sensing assembly is provided with an electromagnetic interference (EMI) shield with at least one aperture configured for allowing the passage of air to said at least one gas sensor. The housing is provided with at least one first opening for allowing an air flow towards the at least one gas sensor and the particle counter, and a second opening connected to an exhaust outlet of the particle counter at a distance of said at least one first opening.

The housing formed by the support core and the cover assembly provides a protection against external elements such as dirt and water, whilst at the same time allowing that new (i.e. not yet analysed) air flows towards the air quality sensing assembly, and that air that has been analysed in the particle counter and that flows out of the exhaust outlet of the particle counter can flow out of the housing. By providing the second opening at a distance of the at least one first opening, the risk that air that has already been analysed in the particle counter, flows back into the housing, is reduced. Further, by providing an EMI shield with at least one aperture configured for allowing the passage of air to said at least one gas sensor, the risk that the measurements of the at least one gas sensor are disturbed by electromagnetic waves, is reduced, whilst at the same time allowing that new air flows towards the at least one gas sensor.

Preferred embodiments thereof are disclosed in the dependent claims.

The top end may be an open end configured for connection, preferably in a tight manner, to an open bottom end of a pole module above or for connection to a cap. Alternatively the top end may be a closed end configured to be used as an upper end of a lamp post, e.g. when the air quality pole module is intended to be used as an upper pole module. The bottom end may be configured for connection to a pole module below or to a support pole below. Preferably, the bottom end is an open end configured for connection, preferably in a tight manner, to an open top end of a pole module below.

According to an exemplary embodiment a power line for feeding the control unit passes through the bottom end of the core support. Further a data line for communicating data from or to the control unit may pass through the bottom end and/or the top end of the core support. The term line has to be interpreted broadly and includes cables and/or electronical or electrical connectors.

According to an exemplary embodiment the EMI shield comprises an air inlet face which is oriented under an angle with respect to the axial direction, in a range from 10° up to and including 90°, preferably from 20° up to and including 80°, said air inlet face comprising said at least one aperture configured for allowing the passage of air to said at least one gas sensor. The at least one gas sensor is arranged on an upper side of the air inlet face of the shield, wherein optionally one or more intermediate layers may be inserted between the EMI shield and the at least one gas sensor. By arranging the at least one aperture in an horizontal or inclined air inlet face of the EMI shield, with the at least one gas sensor arranged above said air inlet face, the risk of negatively influencing the measurements by water or dirt infiltrating in the housing of the pole module, is reduced.

According to an exemplary embodiment the EMI shield may consist of a plate. In other embodiments the EMI shield may have a shell shape or may be a casing, depending on the required degree of EMI shielding.

According to an exemplary embodiment the at least one first opening and/or the second opening are arranged in the cover assembly. In that manner existing support cores (typically made from a rigid material such as metal) can be used without the need for providing extra apertures therein.

According to an exemplary embodiment the at least one first opening of the housing comprises at least five apertures, preferably at least ten apertures, more preferably at least 20 apertures. In that manner the apertures can be relatively small (and still ensure a sufficiently large air flow), reducing the amount of external elements like dirt or water which may infiltrate through the apertures. Preferably the apertures are arranged in a cover section of the cover assembly. Preferably, the cover section is cylindrical, and the apertures are arranged according to a pattern extending over a sufficiently large surface, e.g. over a surface describing an angle of at least 90°, preferably at least 120° around the axial direction of the pole module. The apertures may be holes, such as round holes.

According to an exemplary embodiment the second opening in the housing, which is connected to the exhaust outlet of the particle counter, is arranged in a bottom part of the housing, preferably in a bottom part of the cover assembly, below the at least one first opening, seen in the axial direction of the pole module. In that manner air that has been analysed is evacuated at the bottom while new air can enter through the at least one first opening which is located in a higher position.

According to an exemplary embodiment the at least one gas sensor is configured to sense a gas concentration measure for at least one of the following air pollutants: NO₂, O₃, NO, CO, SO₂, H₂S. Optionally the air quality sensing assembly may further comprise any one or more of the following: a temperature sensor, a humidity sensor, a photo-ionization detector configured to measure volatile organic compounds, an infrared sensor configured to measure a CO₂ concentration.

According to an exemplary embodiment the control unit is configured for outputting to another pole module of the lamp post a feedback signal based on the obtained air quality data. According to another exemplary embodiment the air quality pole module further comprises an output interface configured for outputting information to a user based on the obtained air quality data. In other words, a user may be informed about the quality of the air, either through the air quality pole module itself or through another pole module of the lamp post. The output interface may comprise e.g. any one or more of the following: a light source such as a light source capable of emitting light in different colours based on the obtained air quality data or capable of changing a lighting pattern in time or space in function of the obtained air quality data; a display, e.g. a display configured to display measured values of the air quality; an audio interface configured to emit an audio signal based on the obtained air quality data, etc.

According to yet another embodiment the control unit is configured for transmitting a feedback signal based on the obtained air quality data to a remote server which may communicate the received data to users, e.g. the server may send warning messages to mobile devices of persons in the area of the lamp post. Also a mobile device of a user could be provided with a suitable air quality app capable of receiving air quality data from the server or directly from the air quality module of the lamp post. Also, a user could be a computer device of the municipalities, which is configured to regulate the traffic based on the obtained air quality data. For example, the municipalities could take appropriate measures such as a speed limitation for vehicles, a driving prohibition e.g. for diesel cars, for cars with an odd/pair car plate number, etc.

According to an exemplary embodiment the connection between the second opening and the exhaust outlet of the particle counter is sealed; the cover assembly is attached to the core support in a sealed manner; and the EMI shield is attached to the cover assembly in a sealed manner.

According to an exemplary embodiment the cover assembly comprises a plurality of sections such that an air inlet compartment is formed between the at least one first opening, on the one hand, and the at least one aperture and an air intake of the particle compartment, on the other hand.

According to another aspect there is provided a lamp post comprising an air quality pole module according to any one of the embodiments disclosed above.

According to an exemplary embodiment the lamp post further comprises a support pole and a light pole module comprising a light source; wherein the light pole module and the air quality pole module are arranged in any order one above the other, aligned with the support pole. The lamp post may further comprise any one of the following: an antenna pole module, a base station module comprising base station circuitry; a loudspeaker pole module, a camera pole module, a signal pole module (e.g. light ring module), etc.

According to an exemplary embodiment the control unit of the air quality pole module is configured for outputting a feedback signal based on the obtained air quality data, to another pole module of the lamp post; wherein said other pole module comprises an output interface configured for informing a user based on said feedback signal. The output interface may comprise any one or more of the following: a light source such as a light source capable of emitting light in different colours based on the received feedback signal or capable of changing a lighting pattern in time or space in function of the received feedback signal; a display, e.g. a display configured to display measured values of the air quality; an audio interface configured to emit an audio signal based on the received feedback signal, etc.

### BRIEF DESCRIPTION OF THE FIGURES

The accompanying drawings are used to illustrate presently preferred non-limiting exemplary embodiments of devices of the present invention. The above and other advantages of the features and objects of the invention will become more apparent and the invention will be better understood from the following detailed description when read in conjunction with the accompanying drawings, in which:

FIG. 1 illustrates schematically an exemplary embodiment of a lamp post of the invention;

FIG. 2 illustrates schematically an exploded perspective view of an exemplary embodiment of an air quality pole module for insertion in a lamp post;

FIG. 3 illustrates a schematic cross section of an exemplary embodiment of an air quality pole module;

FIG. 4 illustrates a schematic cross section of another exemplary embodiment of an air quality pole module;

FIGS. 5A and 5B illustrate schematically how two pole modules can be connected to each other; and

FIGS. 6A and 6B illustrate a schematic sectional view and a perspective view of another exemplary embodiment of an air quality pole module, respectively.

### DESCRIPTION OF EMBODIMENTS

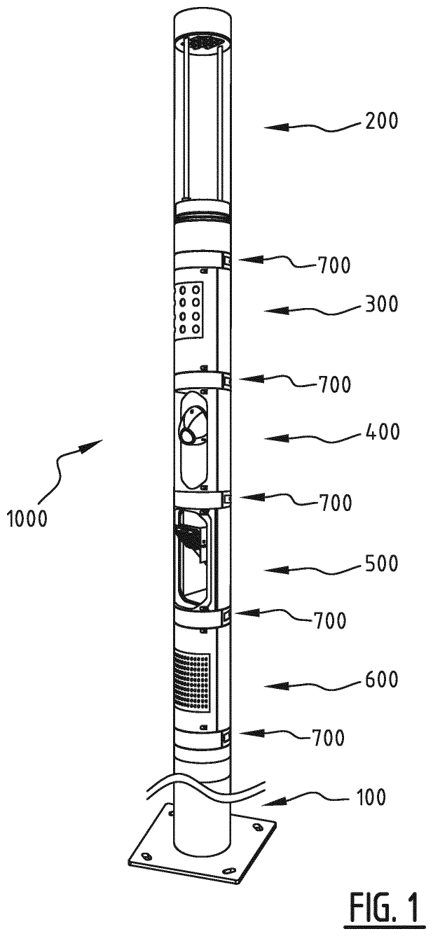

FIG. 1 illustrates schematically an exemplary embodiment of a lamp post 1000. The lamp post 1000 comprises a support pole 100 and a plurality of pole modules 200, 300, 400, 500, 600 supported by the support pole 100. In the illustrated embodiment the plurality of pole modules comprises a light pole module 200 comprising a light source, an air quality pole module 300, a camera pole module 400, a further light pole module 500 and a loudspeaker pole module 600. The support pole 100 may be hollow, and may be provided with a removable door providing access to an inner part of said support pole 100. Further a signal pole module (not shown), such as a light ring module may be included in the lamp post.

It is noted that the term “supported” as in “the light pole module is supported by the support pole” and “the air quality pole module is supported by the support pole” does not imply that the light pole module needs to be directly fixed on the support pole; indeed, there may be intermediate pole modules or elements between the support pole and the light pole module and/or between the support pole and any other pole module; the support pole supports the light pole module, the air quality pole module and any intermediate modules or elements.

Other examples of functionalities which may be included in one or more pole modules and/or in the support pole are any one or more of the following:
an antenna configured for receiving and emitting cellular data;
power management circuitry comprising e.g. one or more of: a power meter, a fuse, a line protection, a circuit breaker, an electrical connection for multiple power lines, a clock, an astroclock, a power supply module, an PLC, a computer, a communication module, display circuitry, etc.; preferably the power management circuitry is configured to manage the provision of power to one or more lamp posts, preferably at least three lamp posts, e.g. more than ten lamp posts. In such embodiments power connection cables pass from the respective pole module through the support pole to other lamp posts, e.g. underground;
telecommunication circuitry for wired or wireless communication, which can comprise at least one of: an optical fibre connection, a fibre to copper interface, a fibre patch panel, a modem, a router, a switch, a patch panel, a network video recorder (NVR), a computer;
audio system management circuitry which can comprise at least one of: an amplifier, a transformer, a media player (connected to network or not), electrical connections for multiple loudspeaker lines, a computer;
WiFi circuitry;
charger circuitry, e.g. phone/computer/tablet charger circuitry or vehicle charger circuitry; or UAV charger circuitry (e.g. drone charger circuitry);
a sound sensor, a microphone, a voice recorder, a detector of smoke, an image sensor, etc., and the associated circuitry;
any human interface device (HID) and the associated circuitry, e.g. a camera, a loudspeaker, a button, a display, etc.
a signaling device, e.g. a light ring capable of performing signaling;
a mechanical and/or electrical plug-in device, e.g. a universal plug-in module, e.g. a mechanical device to fix a flag, a waste bin, etc.; a socket plug-in device.

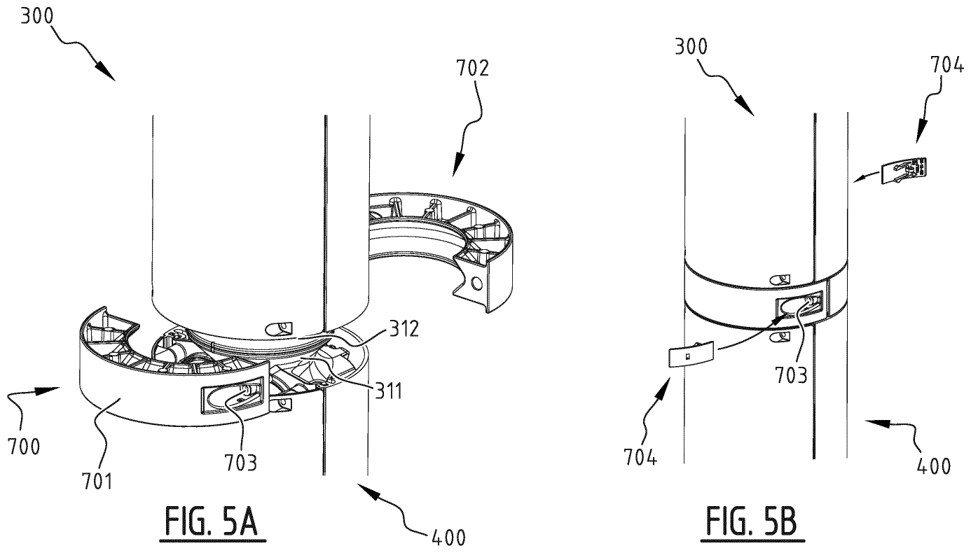

The modules 200, 300, 400, 500, 600 may be arranged in any order one above the other, and may be connected to the support pole 100 and to each other in any suitable way, e.g. using pole module connectors 700 as described in EP 3 076 073 B1 in the name of the applicant which is included herein by reference. FIGS. 5A and 5B illustrate in detail how two pole modules may be connected to each other using a pole module connector 700 comprising two connecting portions 701, 702 which can be clamped around round end parts 311, 312 of the pole modules/support pole. A pole module 300 can be rotated around the axial direction A of the support pole 100 in a desired position and then fixed by the connecting portions 701, 702 and a fixation means 703 for coupling the two connecting portions 701, 702 to each other around round end parts 311, 312 of the pole modules/support pole to be connected. Optionally cover means 704 to hide the fixation means 703 may be attached to (e.g. snapped onto) the connecting portions 701, 702.

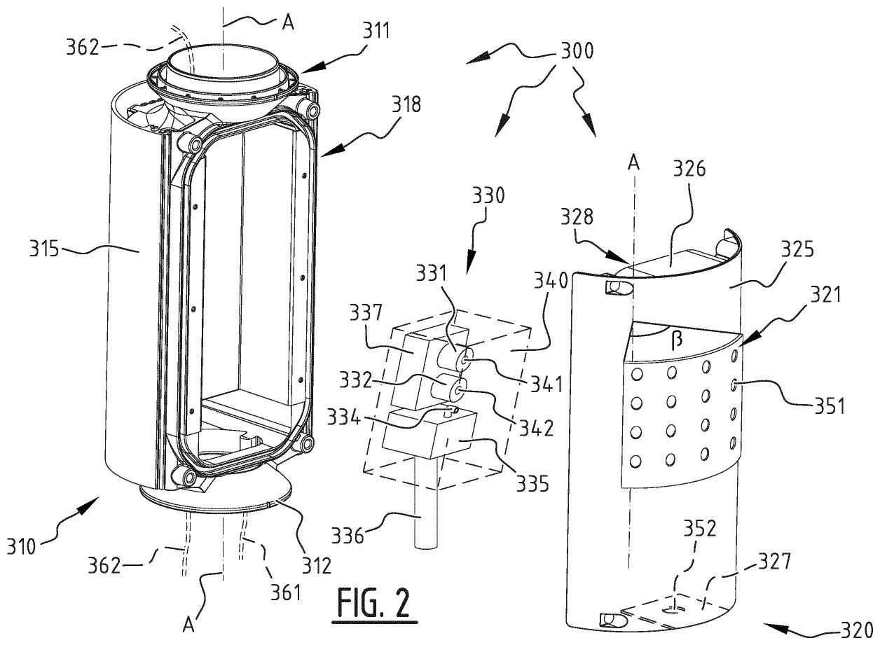

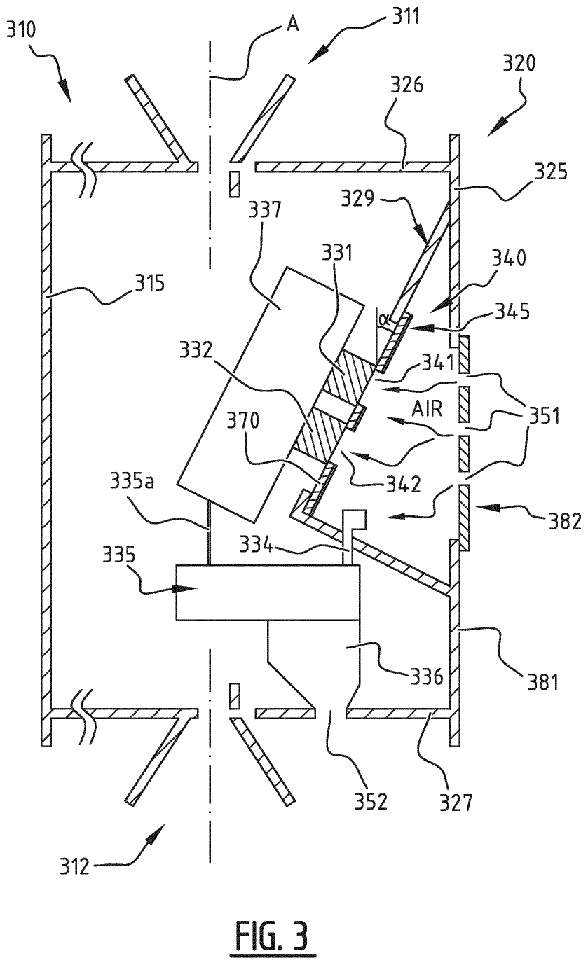

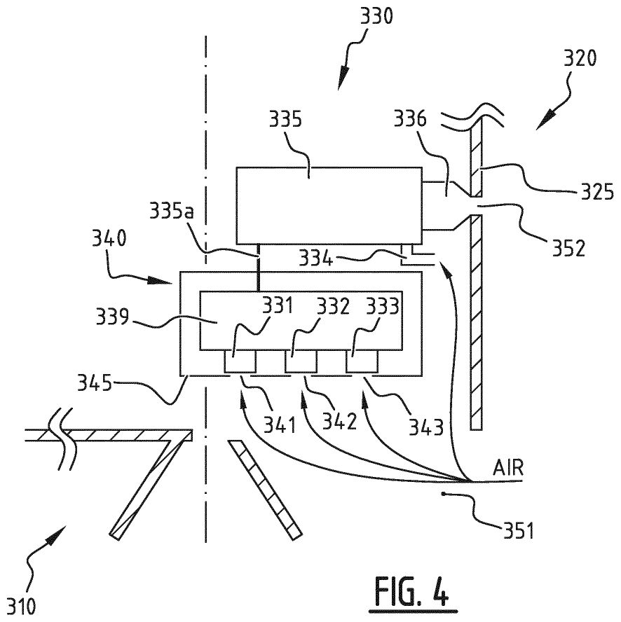

As illustrated in FIG. 1, the air quality pole module 300 is intended for insertion in a lamp post 1000. The air quality pole module 300 is illustrated in detail in FIG. 2, and comprises a core support 310, a cover assembly 320, and an air quality sensing assembly 330. The air quality sensing assembly 330 is schematically illustrated as a block in which a plurality of schematically represented components are arranged, and more detailed exemplary embodiments will be illustrated below with reference to FIGS. 3 and 4. The core support 310 extends in the axial direction A of the pole module which corresponds with an axial direction of the lamp post 1000. The core support 310 has a top end 311 for connection to a pole module above or to a cap, and a bottom end 312 for connection to a pole module below or for connection to the pole support 100. In the illustrated embodiment the top end 311 is an open end (allowing the passage of electrical/data lines from one pole module to the next) configured for connection, preferably in a tight manner, to an open bottom end of a pole module above or for connection to a cap. Alternatively the top end 311 may be a closed end configured to be used as an upper end of the lamp post 1000, e.g. when the air quality pole module is intended to be used as an upper pole module in a way similar to upper module 200 in FIG. 1. In the illustrated embodiment, the bottom end 312 is an open end configured for connection, preferably in a tight manner, to an open top end of a pole module/pole support below. It is noted that the tight connection is typically obtained by a gasket (not shown) positioned between the bottom end 312 and the top end 311 of a pole module below. The connecting portions 701, 702 (see FIGS. 5A and 5B) are arranged in a non-air-tight manner around a bottom and top end 311, 312 to be connected.

In the mounted state, the cover assembly 320 is coupled to the core support 310, such that the core support 310 and the cover assembly 320 form a housing for the air quality sensing assembly 330.

The core support 310 is provided with a circumferential edge 318 which is fixed against a corresponding circumferential edge 328 of the cover assembly 320, wherein preferably a gasket is arranged between the circumferential edges 318, 328 to obtain an appropriately sealed housing.

The air quality sensing assembly 330 comprises at least one gas sensor 331, 332, a particle counter 335 and a control unit 337 for obtaining and outputting air quality data based on measurements by said at least one gas sensor 331, 332 and the particle counter 335. The air quality sensing assembly 330 is provided with an EMI shield 340 with at least one aperture 341, 342 configured for allowing the passage of air to the at least one gas sensor 331, 332.

The housing formed by the core support 310 and the cover assembly 320 is provided with at least one first opening 351 for allowing an air flow through the at least one aperture 341, 342 towards the at least one gas sensor 331, 332 and towards the particle counter 335. The housing is further provided with a second opening 352 connected to an exhaust outlet 336 of the particle counter 335. The second opening 352 is located at a distance of the at least one first opening 351. In the illustrated embodiment, the first and second openings 351, 352 are provided in the cover assembly 320. However, the skilled person understands that e.g. the second opening 352 could also be provided in the support core 310. According to another possibility, both the openings 351 and 352 are provided in the support core 310. According to yet another embodiment, the at least one first opening 351 is provided in the support core 310, and the second opening 352 is provided in the cover assembly 320.

The function of the housing formed by the support core 310 and the cover 320 is to provide a protection against external elements such as dirt and water, whilst at the same time allowing that air flows towards the air quality sensing assembly 330, and that analysed air coming out of the particle counter 335 can flow out of the housing. By providing the second opening 352 at a distance of the at least one first opening 351, it is ensured that air flowing out of the particle counter 335 through an exhaust outlet 336 does not flow back into the housing, as this would disturb the measurements. Further, by providing an EMI shield 340 with at least one aperture 341, 342, it is avoided that the circuitry of the air quality sensing assembly 330 is significantly disturbed by electromagnetic waves, whilst at the same time allowing that air flows towards air intakes of the at least one gas sensor 331, 332 and an air intake 334 of the particle counter 335.

A power line 361 for feeding the control unit 337 passes through the bottom end 312 of the core support 310. The power line 361 may include one or more cable sections and/or one or more connection pieces. Further, a data line 362 for communicating data from or to the control unit 330 may pass through the bottom end 312 and/or through the top end 311 of the core support 310. Alternatively, the control unit 330 may be provided with a wireless communication interface for communicating the air quality data wirelessly.

The control unit 337 of the air quality pole module 300 is connected to the at least one gas sensor 331, 332 and to the particle counter 335 (see connection line 335a) for obtaining the measured data. The control unit 337 may be further configured for outputting a feedback signal based on the obtained air quality data, to another pole module of the lamp post, wherein the other pole module comprises an output interface configured for informing a user based on the feedback signal.

Alternatively such an output interface (not shown) may be integrated in the air quality pole module 300 itself. The output interface may comprise any one or more of the following: a light source such as a light source capable of emitting light in different colours or capable of changing a lighting pattern in time or space (e.g. the lighting pattern may be such that a signal, letter, number or any other indication is projected on the ground or on a wall); a display, e.g. a display configured to display measured values of the air quality; an audio interface configured to emit an audio signal, etc. Such a light source may be part of a signal pole module, e.g. a light ring pole module comprising a ring shaped light source.

In the illustrated embodiment of FIG. 2, the cover assembly 320 comprises a cylindrical cover section 321 which is provided with one or more of first openings 351 arranged according to a pattern. Typically, a plurality of first openings 351 is provided, preferably in the form of apertures. The number of apertures 351 of the pattern may be more than 10, preferably more than 20. The pattern is such that it extends over a cylindrical surface section having an angle β of at least 90°, preferably at least 120° around the axial direction A of the pole module. In that manner a good air flow towards the air quality sensing assembly can be guaranteed, independent of the direction of the wind. Further, by incorporating a large number of apertures 351 in the cover assembly, the apertures can be relatively small such that the amount of external elements such as water or dirt, which enter the housing can be reduced. The second opening 352 which is connected to the exhaust outlet 336 of the particle counter 335, preferably in a tight manner, is arranged in a bottom part of the cover assembly 320, below the plurality of first openings 351, seen in the axial direction A of the pole module. In the illustrated embodiment, the cover assembly 320 comprises a cylindrical section 325 centred around the axial direction A, a bottom section 327 perpendicular on the axial direction A, and a top section 326 perpendicular on the axial direction A. In the illustrated embodiment, the second opening 352 is provided in the bottom section 327, but the skilled person understands that it could also be provided e.g. in a lower area of the cylindrical section 325. According to an alternative embodiment, the second opening 352 could be provided above the one or more first openings 351, as in the embodiment of FIG. 4 which will be discussed below. According to yet another variant, the one or more first openings 351 could be provided on one side of the cover assembly 320, and the second opening 352 could be provided on the other side of the cover assembly 320, e.g. on a left and right side of the cover.

As explained above the tight connection between two superposed pole modules, or between a pole module superposed to the support pole, is typically obtained by a gasket (not shown) positioned between the bottom end 312 of a pole module above and the top end 311 of a pole module/support pole below. The connecting portions 701, 702 (see FIGS. 5A and 5B) are arranged in a non-air-tight manner around a bottom and top end 311, 312 to be connected. Thus, the analysed air escaping through the second opening 352 in the bottom section 327 can escape through the connecting portions 701, 702.

FIG. 2 illustrates an embodiment with two gas sensors 331, 332. However, the skilled person understands that more or less than two gas sensors may be provided, to sense a gas concentration measure for one or more of the following air pollutants: NO₂, O₃, NO, CO, SO₂, H₂S. The gas sensors may be any suitable commercially available gas sensor. Also, the particle counter 335 may be any suitable commercially available particle counter, e.g. an optical particle counter (also called detector) configured to measure particle matter (e.g. PM_{1.0}, PM_{2.5}, PM₁₀). Also, the air quality sensing assembly 330 may comprise any one or more of the following: a temperature sensor, a humidity sensor, a photo ionisation detector configured to measure volatile organic compounds, an infrared sensor configured to measure a CO₂ concentration.

The core support 310 is provided with an outer section having a first cylindrical outer surface 315. As explained above, the cover assembly 320 also has a cylindrical outer surface 325. The cylindrical outer surfaces 315, 325 together describe a complete cylindrical outer surface having an axis corresponding with the axial direction A of the pole module. The top and bottom end 311, 312 of the core support 310 each comprise a ring section having an axis corresponding with the axial direction A of the pole module. In that manner, cable and/or power lines may go from one pole module to the next. Preferably, the core support 310 is made of metal, such that a rigid core support structure is obtained capable of supporting one or more further pole modules on top of the air quality pole module 300. The cover assembly 320 may be made fully or partially from plastic and/or from metal.

Preferably, the air quality pole module 300 is arranged in the lamp post 1000 at a height between 3 and 4 metres above the ground. FIG. 1 illustrates an embodiment where the air quality pole module 300 is arranged below the light pole module 200, but the skilled person understands that it may also be mounted above the light pole module 200.

FIG. 3 illustrates in more detail a cross section of an exemplary embodiment of an air quality pole module 300. In this embodiment the EMI shield 340 may comprise an air inlet face 345 which is oriented under an angle a with respect to the axial direction A. Preferably, the angle is in a range going from 10° up to and including 90°, more preferably from 20° up to and including 80°. The air inlet face 345 may be inclined, running upwardly in the direction of the cover assembly 320. Alternatively the air inlet face 345 may be oriented horizontally, with the gas sensors 331, 332 located above the air inlet face 345 as illustrated in FIG. 4. In that manner, even if external elements such as moisture and/or dirt enter through the cover assembly 320, the amount thereof that disturbs the air quality sensing assembly 330 can be limited. The air inlet face 345 comprises the at least one aperture 341, 342 configured for allowing the passage of air to the at least one gas sensor 331, 332. The gas sensors 331, 332 may be arranged on a PCB which is part of the control unit 337. The control unit 337 may comprise both analog and/or digital circuitry as well as a microprocessor for obtaining and processing the sensor data, and for outputting air quality data based on the sensor data. The skilled person understands that more or less control circuitry may be provided in the control unit 337 of the air quality pole module, and that the processing of the measured data may also be done partially or fully remotely, e.g. in a cloud computing environment or on one or more remote servers or in processing circuitry in another pole module. However, typically the control unit will comprise a PCB on which the gas sensors 331, 332 are arranged and some circuitry, e.g. analogue to digital convertor circuitry and processing circuitry. For example, the control unit 337 may be a commercially available Raspberry Pi controller with Ethernet, USB and HDMI connectors. The particle counter 335 may have a separate air intake 334 arranged to receive air which flows through the at least one opening 351 in the cover assembly 320. Further, the particle counter 335 has an exhaust outlet 336 leading to the second opening 352 in the cover assembly 320.

The cover assembly 320 illustrated in FIG. 2 comprises sections 325, 326 and 327 which have been described above and a mounting section 329 against which the air inlet face 345 of the EMI shield 340 is fixed, preferably in a tight manner. This may be achieved by inserting a gasket 370 between the air inlet face 345 and the mounting section 329. The gasket 370 may be a sheet gasket in which apertures have been arranged corresponding to the apertures 341, 342 in the EMI shield 340. In the embodiment of FIG. 3 cylindrical section 325 comprises two parts 381, 382, wherein part 381 is fixed to part 382. This is convenient for mounting purposes. Further air intake 334 of particle counter 335 protrudes through the mounting section 329, preferably in a sealed manner. By having a mounting section 329 shaped as in FIG. 3 an air intake compartment is created between the first opening 351, on the one hand, and the apertures 341, 342 and the air intake 334, on the other hand.

FIG. 4 illustrates a cross section of another exemplary embodiment of an air quality pole module 300. In this embodiment the EMI shield 340 is a metal casing with an air inlet face 345 which is oriented more or less horizontally, with three gas sensors 331, 332, 333 located above the air inlet face 345. Typically only the gas sensors 331, 332, 333 are EMI sensitive. However, as it is preferred to mount the control unit 337 close to the gas sensors 331, 332, 333, it may be advantageous to enclose both the gas sensors 331, 332, 333 and the control unit 337 in the metal casing 340 as shown in FIG. 4. The control unit 337 is connected (see connection line 335a) to a particle counter 335 arranged outside the metal casing 340. In this embodiment, the particle counter 335 may be arranged above the gas sensors 331, 332, 333, and is provided with an exhaust outlet 336 which leads to an opening 352 in the cover assembly 320, here in a vertical outer wall 325 of the cover assembly 320. In the embodiment of FIG. 4 the first opening 351 is provided at the bottom of the cover assembly 320, e.g. by having an open bottom section instead of having a closed section 327 as in FIG. 3 (with a small second opening 352 in the bottom section 327 in FIG. 3). In FIG. 4 the cover assembly 320 is shown with only an outer section 325, but the skilled person understands that additional sections such as a suitable mounting section may be included in order to obtain the required tightness of the housing. Further gaskets may be added where required.

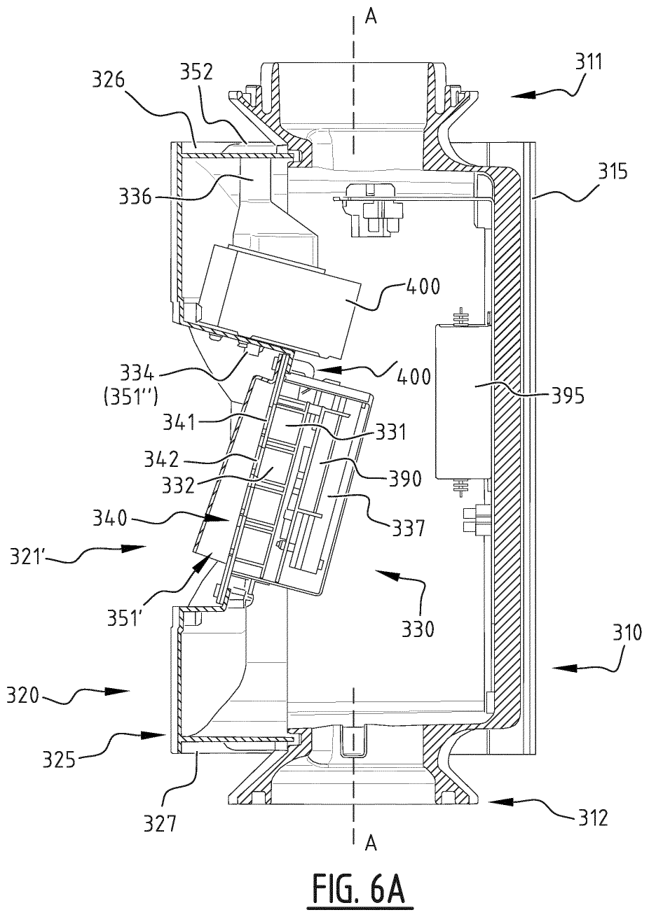

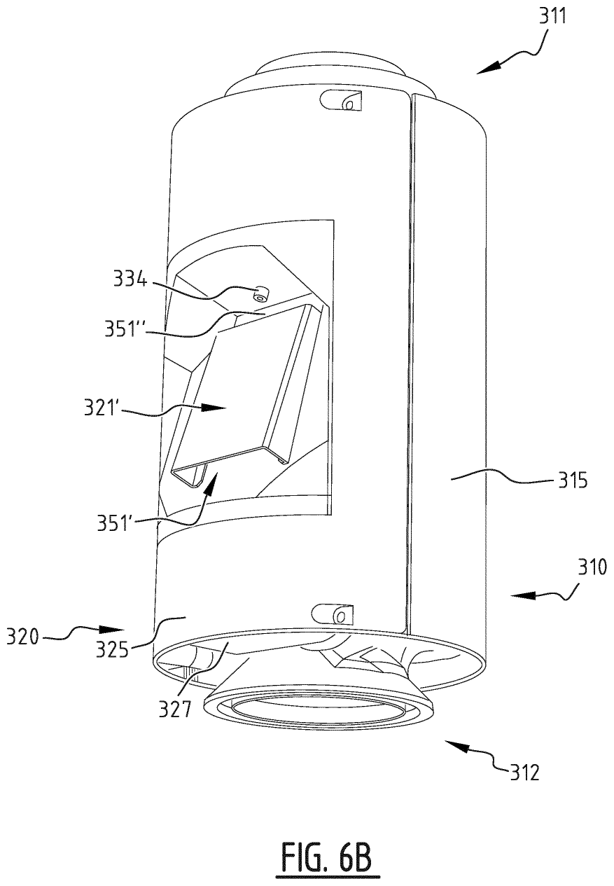

FIGS. 6A and 6B illustrate a variant of the exemplary embodiment of FIG. 1, wherein the same reference numerals have been used to refer to the same or similar parts. The air quality pole module 300 comprises a core support 310, a cover assembly 320, and an air quality sensing assembly 330. The core support 310 extends in the axial direction A of the pole module which corresponds with an axial direction of the lamp post 1000. The core support 310 has a top end 311 for connection to a pole module above or to a cap, and a bottom end 312 for connection to a pole module below or for connection to the pole support 100. In the illustrated embodiment the top end 311 is an open end (allowing for the passage of electrical/data lines from one pole module to the next) configured for connection, preferably in a tight manner, to an open bottom end of a pole module above or for connection to a cap. Alternatively the top end 311 may be a closed end configured to be used as an upper end of the lamp post 1000. In the illustrated embodiment, the bottom end 312 is an open end configured for connection, preferably in a tight manner, to an open top end of a pole module/pole support below. In the mounted state, the cover assembly 320 is coupled to the core support 310, such that the core support 310 and the cover assembly 320 form a housing for the air quality sensing assembly 330.

The air quality sensing assembly 330 comprises at least one gas sensor 331, 332, a particle counter 335 and a control unit 337 for obtaining and outputting air quality data based on measurements by said at least one gas sensor 331, 332 and the particle counter 335. Optionally, a holder 390 for a battery (not shown) may be provided adjacent the control unit 337. In addition or alternatively, a power supply unit 395 for the control unit 337 may be arranged, preferably in the housing formed by the core support 310 and the cover assembly 320. The power supply unit 390 may also be provided outside the housing, e.g. in another pole module. The air quality sensing assembly 330 is provided with an EMI shield 340 with at least one aperture 341, 342 configured for allowing the passage of air to the at least one gas sensor 331, 332.

The particle counter 335 may have a separate air intake 334 arranged to receive air which flows through an opening 351″ in the cover assembly 320. Further, the particle counter 335 has an exhaust outlet 336 leading to a second opening 352 in the cover assembly 320.

The cover assembly 320 may comprise a cylindrical cover section 321 (omitted in FIG. 6A and 6B, but shown in FIGS. 2 and 3) which is provided with one or more of openings 351 (as in FIGS. 2 and 3) arranged according to a pattern as has been described above in connection with FIGS. 2 and 3. In addition, the cover assembly 320 comprises a cover section 321′ arranged to cover the at least one aperture 341, 342, whilst allowing air to pass through an opening 351′ at a lower end of cover section 321′. The cover section 321′ protects the at least one aperture 341, 342 against e.g. water and dust. This cover element 321′ may be made with a folded metal plate, such as an aluminium plate. In another embodiment, it could be a plastic element. More generally, any suitable cover section 321′ may be used. Optionally, in the embodiment of FIG. 6A and 6B, the outer cover section 321 (not shown) may be omitted. In that case the at least one first opening consists of opening 351′ leading to the at least one aperture 341, 342, and opening 351″ forming the inlet of the air intake 334. The second opening 352 which is connected to the exhaust outlet 336 of the particle counter 335, preferably in a tight manner, is arranged in an upper part of the cover assembly 320, above the one or more first openings 351′, 351″, seen in the axial direction A of the pole module. In the illustrated embodiment, the cover assembly 320 comprises a cylindrical section 325 centred around the axial direction A, a bottom section 327 perpendicular on the axial direction A, and a top section 326 perpendicular on the axial direction A. In the illustrated embodiment, the second opening 352 is provided in the top section 326, but the skilled person understands that it could also be provided e.g. in an upper area of the cylindrical section 325. As explained the connecting portions 701, 702 (see FIGS. 5A and 5B) are arranged in a non-air-tight manner around a bottom and top end 311, 312 to be connected. Thus, the analysed air escaping through the second opening 352 in the top section 326 can escape through the connecting portions 701, 702.

The gas sensors 331, 332 may have any one or more of the features described above in connection with the other embodiments, and optionally other sensors may be provided as described above.

The cylindrical outer surfaces 315, 325 together may describe a complete cylindrical outer surface having an axis corresponding with the axial direction A of the pole module. The top and bottom end 311, 312 of the core support 310 may each comprise a ring section having an axis corresponding with the axial direction A of the pole module. In that manner, cable and/or power lines may go from one pole module to the next. Preferably, the core support 310 is made of metal, such that a rigid core support structure is obtained capable of supporting one or more further pole modules on top of the air quality pole module 300. The cover assembly 320 may be made fully or partially from plastic and/or from metal.

The EMI shield 340 may comprise an air inlet face 345 which is oriented under an angle a with respect to the axial direction A. The air inlet face 345 may be vertical (not shown in FIGS. 6A and 6B) or inclined, running upwardly in a direction away from the cover assembly 320 (as shown in FIG. 6A and 6B) or in a direction towards the cover assembly 320 (as shown in FIGS. 2 and 3). The air inlet face 345 comprises the at least one aperture 341, 342 configured for allowing the passage of air to the at least one gas sensor 331, 332. The gas sensors 331, 332 may be arranged on a PCB which is part of the control unit 337. The control unit 337 may be as described above in connection with FIGS. 2 and 3.

The cover assembly 320 illustrated in FIG. 6A comprises sections 325, 326 and 327 which have been described above and a mounting section 329 against which the air inlet face 345 of the EMI shield 340 is fixed, preferably in a tight manner. Further air intake 334 of particle counter 335 protrudes through the mounting section 329, preferably in a sealed manner. The air intake 334 is directed downwardly, such that the air inlet 351″ is protected from water and dust.

The housing formed by the core support 310 and the cover assembly 320 is provided with at least one first opening 351′ for allowing an air flow through the at least one aperture 341, 342 towards the at least one gas sensor 331, 332, and a first opening 351″ leading to the air intake 334. The housing is further provided with a second opening 352 connected to an exhaust outlet 336 of the particle counter 335. The second opening 352 is located at a distance of the at least one first opening 351′, 351″.

The function of the housing formed by the support core 310 and the cover 320 is to provide a protection against external elements such as dirt and water, whilst at the same time allowing that air flows towards the air quality sensing assembly 330, and that analysed air coming out of the particle counter 335 can flow out of the housing. By providing the second opening 352 at a distance of the at least one first opening 351′, 351″, it is ensured that air flowing out of the particle counter 335 through an exhaust outlet 336 does not flow back into the housing, as this would disturb the measurements.

Whilst the principles of the invention have been set out above in connection with specific embodiments, it is to be understood that this description is merely made by way of example and not as a limitation of the scope of protection which is determined by the appended claims.

1. An air quality pole module for a lamp post comprising a plurality of pole modules arranged one above the other in an axial direction, said air quality pole module comprising:
a core support extending in the axial direction between a top end and a bottom end;
a cover assembly attached to the core support such that the core support and the cover assembly together form a housing;
an air quality sensing assembly arranged in said housing and comprising at least one gas sensor, a particle counter and a control unit configured for obtaining and outputting air quality data based on measurements by said at least one gas sensor and the particle counter, wherein the air quality sensing assembly is provided with an electromagnetic interference (EMI) shield with at least one aperture configured for allowing the passage of air to said at least one gas sensor;
wherein the housing is provided with:
at least one first opening for allowing an air flow towards the at least one gas sensor and the particle counter, and
a second opening connected to an exhaust outlet of the particle counter at a distance of said at least one first opening.

2. The air quality pole module according to claim 1, wherein a power line for feeding the control unit passes through the bottom end of the core support.

3. The air quality pole module according to claim 1, wherein a data line for communicating data from or to the control unit passes through the bottom end or the top end of the core support.

4. The air quality pole module according claim 1, wherein the connection between the second opening and the exhaust outlet of the particle counter is sealed, wherein the cover assembly is attached to the core support in a sealed manner, and wherein the EMI shield is attached to the cover assembly in a sealed manner.

5. The air quality pole module according to claim 1, wherein the EMI shield comprises an air inlet face which is oriented under an angle with respect to the axial direction, in a range from 10° up to and including 90°, said air inlet face comprising said at least one aperture configured for allowing the passage of air to said at least one gas sensor.

6. The air quality pole module according to claim 1, wherein the at least one first opening or the second opening is arranged in the cover assembly.

7. The air quality pole module according to claim 1, wherein the at least one first opening of the housing comprises at least five apertures.

8. The air quality pole module according to claim 7, wherein the apertures are arranged in a cover section of the cover assembly.

9. The air quality pole module according to claim 8, wherein the cover section is cylindrical, and wherein the apertures are arranged according to a pattern extending over an angle of at least 90° around the axial direction of the pole module.

10. The air quality pole module according to claim 1, wherein the second opening in the housing, which is connected to the exhaust outlet of the particle counter, is arranged in a bottom part of the housing, below the at least one first opening, seen in the axial direction of the pole module.

11. The air quality pole module according to claim 1, wherein the at least one gas sensor is configured to sense a gas concentration measure for at least one of the following air pollutants: NO₂, O₃, NO, CO, SO₂, or H₂S.

12. (canceled)

13. The air quality pole module according to claim 1, wherein the core support is provided with an outer section having a first outer surface, and wherein the cover assembly has a second outer surface, such that the first and second outer surface together define a cylindrical outer surface centred around the axial direction of the pole module.

14. The air quality pole module according to claim 1, wherein the top and bottom end of the core support each comprise a ring section centred around the axial direction of the pole module.

15. (canceled)

16. The air quality pole module according to claim 1, wherein the control unit is configured for outputting to another pole module of the lamp post a feedback signal based on the obtained air quality data.

17. The air quality pole module according to claim 1, further comprising an output interface configured for outputting information to a user based on the obtained air quality data, wherein the output interface comprises any one or more of the following: a light source, a display, or an audio interface.

18. The air quality pole module according to claim 1, wherein the cover assembly comprises a plurality of sections such that an air inlet compartment is formed between (i) the at least one first opening and (ii) the at least one aperture and an air intake of the particle compartment.

19. A lamp post comprising an air quality pole module according to claim 1.

20. The lamp post according to claim 19, further comprising a support pole and a light pole module comprising a light source, wherein the light pole module and the air quality pole module are arranged in any order one above the other, aligned with the support pole.

21. The lamp post according to claim 19, further comprising any one of the following: an antenna pole module, a base station module comprising base station circuitry, a loudspeaker pole module, or a camera pole module.

22. The lamp post according to claim 19, wherein the control unit of the air quality pole module is configured for outputting a feedback signal based on the obtained air quality data, to another pole module of the lamp post, and wherein said other pole module comprises an output interface configured for informing a user based on said feedback signal, wherein the output interface comprises any one or more of the following: a light source capable of emitting light in different colours based on the received feedback signal, a display, or an audio interface.

23. (canceled)
